(19) United States
(12) Patent Application Publication
(10) Pub. No.: US 2022/0409189 A1
(43) Pub. Date: Dec. 29, 2022

(54) SAMPLE COLLECTION DEVICE WITH REAGENT

(71) Applicant: Viome Life Sciences, Inc., Bellevue, WA (US)

(72) Inventors: Ryan TOMA, Los Alamos, NM (US); Nathan DUVAL, Santa Fe, NM (US); Chandler LAURITZEN, Los Alamos, NM (US); Jason DAVIS, Santa Fe, NM (US); Momchilo VUYISICH, Bothell, WA (US)

(21) Appl. No.: 17/779,364

(22) PCT Filed: Dec. 10, 2020
(86) PCT No.: PCT/US20/64326

§ 371 Date: May 24, 2022

Related U.S. Application Data
(60) Provisional application No. 62/947,299, filed on Dec. 12, 2019.

(51) Int. Cl. A61B 10/00; A61J 19/02; B01L 3/00; A61J 1/12

(52) U.S. Cl. CPC A61B 10/0051; A61J 19/02; B01L 3/502; A61J 1/12; B01L 2300/042

(57) ABSTRACT

A fluid collection system comprises a sealed reagent container that fits inside a sample container and is unsealed by disengaging a seal comprised in a sample container. The sealed reagent container contains a reagent, such as a nucleic acid preservative. The reagent container also includes a sleeve disposed opposite the sealed opening. The sleeve includes a distal opening for receiving a sample and a proximal opening disposed in the sample container for guiding a sample from the distal opening and through the proximal opening into the sample container. Disengaging the seal releases the reagent into the sample container with the sample, where they mix.

### REFERENCE TO RELATED APPLICATIONS

This application claims the benefit of the priority date of U.S. provisional application 62/947,299, filed Dec. 12, 2019, the contents of which are incorporated herein by reference in their entirely.

### BACKGROUND

Commercial analysis of biological samples can involve providing a sample collection kit to a user, such as a customer. The user deposits the biological sample into a sample container, seals the sample container, and returns the sealed container to a facility for analysis. Returning a sample container can involve placing the sample container into a shipping container and providing it to a common carrier for transport to a facility for processing.

In some cases, the biological sample is mixed with a preservative in the container before transmission. Devices that allow for mixing of fluids without a separate fluid-handling step simplify the collection process.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary disengaged sample container.

FIG. 2 shows an exemplary engaged sample container.

FIG. 3 shows a top-down view of an exemplary sleeve attached to a reagent container and having a passage through the sleeve.

FIG. 4 shows a schematic of an exemplary sample container and method of use.

FIGS. 5A and 5B show, respectively, a top-down and bottom-up view of a sample container.

### SUMMARY

A fluid collection system comprises a sealed reagent container that fits inside a sample container and is unsealed by disengaging a seal comprised in a sample container. The sealed reagent container contains a reagent, such as a nucleic acid preservative. The reagent container also includes a sleeve disposed opposite the sealed opening. The sleeve includes a distal opening for receiving a sample and a proximal opening disposed in the sample container for guiding a sample from the distal opening and through the proximal opening into the sample container. Disengaging the seal releases the reagent into the sample container with the sample, where they mix.

In one aspect provided herein is a collection device comprising a first container positioned inside a second container, wherein the first container is releasably sealed by the second container, and wherein the first container comprises a sleeve that fits into an opening in the second container and that is configured to transmit fluid received into the sleeve into the second container, and wherein first container comprises a reagent, e.g., a liquid. In one embodiment when an orifice in the first container is oriented toward gravity, unsealing the first container releases the reagent into the second container. In another embodiment the first container and second container are tube-shaped. In another embodiment the first tube has a tapered end. In another embodiment the sleeve is attached to the tapered end. In another embodiment the first container has an external diameter less than an internal diameter of the second container. In another embodiment the first container has an external diameter of about 10 mm to 20 mm and the second container has an internal diameter of about 15 mm to 30 mm. in another embodiment the sleeve is configured as a funnel. In another embodiment the second container comprises threads configured to screw into a top of the first container. In another embodiment the second container comprises a screw protruding from a bottom the second container, wherein the screw comprises threads configured to screw into internal threads in a top of the first container. In another embodiment the second container comprises a stopper configured to stop an orifice in the first container. In another embodiment the first container has a volume of about 2 ml to about 20 ml and the second container has a volume of about 7 ml to about 45 ml. in another embodiment the sleeve is attached to the first container through ribs, wherein spaces between ribs allow sample deposited into the sleeve to pass into the second container. In another embodiment the second container comprises an open end comprising internal or external threads for sealing with a screw cap. In another embodiment the first container and/or the second container comprise a plastic. In another embodiment the plastic comprises polypropylene or polyethylene. In another embodiment the reagent comprises an RNA preservative. In another embodiment the RNA preservative is selected from one or more of formalin, sulfate (e.g., ammonium sulfate), isothiocyanate (e.g., guanidinium isothiocyanate) and urea.

In another aspect provided herein is a kit comprising: (a) a first container comprising: (i) a first orifice communicating with a first internal space; and (ii) a sleeve attached to the first container at an end opposite the first orifice, and wherein a sample deposited in the sleeve can pass through the sleeve; and (b) a second container comprising: (i) a second orifice communicating with a second internal space; and (ii) a seal disposed within the second internal space, wherein the seal is configured to removably seal the first orifice when engaged therewith.

In another aspect provided herein is a collection device comprising: (a) a first container comprising a first orifice communicating with a first internal space; and a sleeve configured to transmit a sample; and (b) a second container comprising a second orifice communicating with a second internal space, and a seal disposed within the second internal space; wherein the first container has dimensions to both fit into the second orifice and be sealed by the seal, and to position the sleeve to transmit fluid received into the sleeve into the second container. In one embodiment the sleeve is configured as a funnel having a wider end and a narrower end, wherein the narrower end is positioned proximal to the second orifice and the wider end is positioned distal to the orifice.

In another aspect provided herein is a kit comprising: (a) a first container comprising: (i) a first container comprising a first orifice communicating with a first internal space; and (ii) a sleeve attached to the first container at an end opposite the first orifice, and wherein a sample deposited in the sleeve can pass through the sleeve; and (b) a second container comprising: (i) a second orifice communicating with a second internal space; (ii) a seal disposed within the second internal space, wherein the seal is configured to removably seal the first orifice when engaged therewith; and wherein the first container is configured to fit within the second container such that: (1) the first orifice mates with and is removably sealed by the seal; (2) the sleeve fits within the second orifice; and (3) fitting the first container into the second container leaves space in the second internal space to hold sample deposited into the sleeve. In one embodiment the kit comprises a second cap configured to close the second orifice. In another embodiment the seal engages the first container within the second internal space. In another embodiment the seal does not engage the first container outside of the first internal space. In another embodiment the seal is positioned distal from the second orifice.

In another aspect provided herein is a method comprising: a) providing a collection device as described herein; b) depositing a sample into the second container through the sleeve; and c) unsealing the first container from the second container, thereby contacting the reagent with the sample. In another embodiment the sample comprises saliva. In another embodiment depositing comprises spitting. In another embodiment the method further comprises, after unsealing, sealing the second container. In another embodiment sealing the second container comprises attaching a screw cap to a top of the second container. In another embodiment after sealing the second container, mixing the reagent and the sample in the second container. In another embodiment the method comprises, after sealing the second container, depositing the second container in a mailing container and transmitting the mailing container via common carrier.

In another aspect provided herein is a method comprising: (a) depositing a reagent through an orifice into an internal space of a first container; and (b) sealing the orifice with a seal formed in an internal space of a second container; wherein the second container has a greater volume than the first container. In one embodiment the reagent comprises a liquid, e.g., an aqueous liquid. In another embodiment the reagent comprises an RNA preservative. In another embodiment the first container has a volume between about 2 ml to about 20 ml. in another embodiment the second container has a volume between about 7 ml to about 45 ml. in another embodiment the seal comprises a screw, and sealing comprises screwing the second container into the orifice of the first container. In another embodiment the seal comprises a stopper, and sealing comprises inserting the stopper into the orifice of the first container. In another embodiment the seal comprises a snap cap, and sealing comprises snapping the cap over the orifice of the first container. In another embodiment the first container and the second container comprise cylindrical portions, and sealing comprises sliding the cylindrical portion of the second contain over the cylindrical portion of the first container. In another embodiment the first container further comprises a sleeve disposed behind the orifice on the first container, wherein the sleeve has openings at both ends and, wherein sealing further comprises inserting a proximal end of the sleeve into an orifice of the second container, such that an opening in a distal end of the sleeve communicates through an opening in the proximal end of the sleeve with an internal space of the second container. In another embodiment the sleeve has a funnel shape with a narrower end positioned proximal to the first container and the wider end positioned distal to the first container. In another embodiment the seal engages the first container within the internal space of the first container. In another embodiment the seal does not engage the first container outside of the internal space of the first container. In another embodiment the seal is positioned distal from an opening to the internal space of the second container.

In another aspect provided herein in a collection device comprising: (a) a first container comprising: (i) at a first end, an opening to a cavity, wherein the cavity contains a liquid; and (ii) at a second end, a funnel-shaped sleeve, wherein the sleeve has a wider mouth and a narrower mouth, and is attached to the first container by one or a plurality of ribs; and (b) a second container comprising: (i) an opening to a cavity, wherein the cavity comprises a cap; wherein: (I) the first container is positioned within the cavity of the second container; (II) the cap releasably closes the opening of the first container; and (III) the narrower mouth of the sleeve is positioned within the opening of the second container and the wider mouth of the sleeve is positioned outside of the opening of the second container.

### DETAILED DESCRIPTION

Disclosed herein is a sample collection device and method of use. The sample collection device is configured to allow collection of a sample, e.g., a fluid, such as saliva, into a container, and mixing with a reagent, e.g., a fluid, such as a preservative, without the need to introduce the reagent into from outside the container. Furthermore, the device is configured such that releasing the reagent into contact with the sample involves disengaging parts of the device (e.g., opening or pulling apart), rather than engaging parts of a device (e.g., closing or puncturing).

The device can include two containers, a sample container and a reagent container. The sample container is wider than the narrower reagent container. Each container is typically configured as a vial or comprising elements of a cylinder. The narrower reagent container slidably fits inside the wider sample container. The sample container contains a sealing mechanism attached to, or integral with, a surface of the internal compartment, typically positioned at the bottom of the compartment into which the reagent container fits. The sealing mechanism is configured to seal the reagent container when the reagent container is inserted into the sample container. The sealing mechanism can include a screw with threads that screw into a top of the reagent container. Alternatively, the sealing mechanism can comprise a stopper that seals the top of the reagent container with a friction fit. Alternatively, the sealing mechanism can comprise a cap with a lip that secures the opening through, for example, a flange. Accordingly, the sample container can function as a “top” or “cap” that both closes the reagent container and that envelops the sides of the reagent container. A sample placed into the reagent container thus can be sealed within it by closing the top of the reagent container with the seal contained in the sample container. Typically, also, the volume of the sample container is greater than volume of the inside container so that the sample container can completely accommodate the reagent sealed in the reagent container with room for a sample.

When the sealed device is oriented with the seal positioned under the reagent in the reagent container, rather than over the reagent container, opening the seal, for example by unscrewing the seal, will release the reagent in the reagent container into the sample container by gravity.

The dimensions of the outside and inside containers are chosen to allow space between the outside wall of the inside container and the inside wall of the outside container. The space is large enough to accommodate a fluid, such as a bodily fluid, such as saliva, to be collected into the lumen of the outside container with the device is sealed.

In certain embodiments, the seal does not comprise a material, such as a film, that, when punctured by a hard or sharp object, breaks the seal. In other embodiments seal does not comprise a twisting mechanism that, upon closing, releases the reagent. In other embodiments seal is not comprise a structure that, when pushed, releases the reagent.

In order to deposit a sample into the sample container, the reagent container is provided with a collection sleeve. The sleeve is attached to the reagent container distal to the opening of the reagent container. The sleeve can be configures as a funnel, that is, a conduit having an opening at a first end that is wider (e.g., greater area) than an opening at a second end (e.g., a mouth (wider) and cloaca (narrower). To accommodate the sleeve, the reagent container can have a tapered end so that the sleeve can fit into the sample container. For example, the sleeve can have a mouth on one side and a drain on the other side. The drain has dimensions to allow it to fit into the opening of the sample container. The sleeve can be attached to the reagent container, for example, by ribs, so that the mouth of the sleeve communicates with the lumen of the sample container through one or more apertures. So, for example, spitting into the mouth of the sleeve can allow saliva to flow through the sleeve and into the sample container.

The dimensions of the sample container can be between about 40 millimeters and about 80 millimeters, e.g., about 60 millimeters, in length and about 10 millimeters to about 20 millimeters, e.g., about 15 millimeters, inside diameter. The seal can be a threaded “bolt” about 15 millimeters to about 25 millimeters, e.g., about 20 millimeters, in length and about 8 millimeters to about 12 millimeters, e.g., about 10 millimeters, in diameter. The dimensions of the reagent container can be about 30 millimeters to about 50 millimeters, e.g., about 40 millimeters, in length and about 8 millimeters to about 12 millimeters, e.g., about 10 millimeters, in diameter. There can be about 2 mm to about 3 mm, e.g., about 2.5 mm of space on either side between the inserted reagent container and sample container.

In order to mix reagent, such as a liquid, contained in the reagent container with a sample introduced through the sleeve into the sample container, the seal is disengaged, for example by unscrewing or uncorking, so the contained liquid falls into the sample container.

A kit can include a cap or seal for the sample container. Once sealed, the sample and reagent can be mixed, for example, by shaking or swirling.

The sample can be any bodily fluid, for example, saliva, urine, blood, breast milk, tears, sweat, etc.

The reagent provided in the reagent container can comprise any liquid, such as an aqueous liquid, e.g., water or a buffered solution. The reagent can further comprise one or more compounds to inhibit or slow degradation of one or more kinds of nucleic acid, such as DNA or RNA, or protein, e.g., a protease inhibitor. As used herein, the term “nucleic acid preservative” refers to a compound or composition that inhibits degradation of nucleic acid. RNA preservatives include, without limitation, formalin, sulfate (e.g., ammonium sulfate), isothiocyanate (e.g., guanidinium isothiocyanate) and urea. Commercially available RNA preservatives include, for example, TRIzol (ThermoFisher), RNAlater (Ambion, Austin, Tex., USA), Allprotect tissue reagent (Qiagen), PAXgene Blood RNA System (PreAnalytiX GmbH, Hombrechtikon), RNA/DNA Shield® (Zymo Research, Irvine, Calif.), and DNAstable (MilliporeSigma, Burlington, Mass.). The reagent can comprise a DNA preservative. The reagent can comprise an enzyme, e.g., a protease. The reagent can comprise an anticoagulant, such as heparin, sodium citrate, potassium oxalate or potassium EDTA. The reagent can comprise a cell-lysis agent. Examples of cell lysis agents include solutions with a salt and an ionic or non-ionic detergent, e.g., Tween-20 or Tween 80, Triton X (e.g., Trigon X 100 or Triton X 114), octyl glucoside or octyl thioglucoside, Brij, SPAN, MEGA, or NP-40, SDS, or ethyl trimethylammonium bromide.

A kit of this disclosure can include each of the reagent and sample containers of a sample collection device as disclosed herein. It can also include a cap or top or lid adapted to close the sample container. In certain embodiments, a kit contains an assembled device in which the reagent container is engaged with the sample container and contains a reagent within. The kit also can include a cap, top or lid for the sample container after disengagement with the reagent container.

A kit can further include a shipping container adapted to hold the sample collection device and to be transmitted to a recipient Such a container is useful for shipping sample-containing sample containers to a facility that is remote from the user's location. As used herein, the term “remote” when referring to a physical location, refers to a location in another building, e.g., located at least any of 1 mile, 10 miles, 100 miles or 1000 miles away or located in another city, state or country. The shipping container can be any container suitable for shipping through a common carrier or private courier to a recipient. For example, a common carrier can be the United States Postal Service, FedEx or UPS. The shipping container can be, for example, an envelope, a bag, a box or a shipping tube. Such shipping containers can have shipping expenses prepaid.

A collection device can be assembled as follows. The compartment of a reagent container can be filled with a reagent, e.g., an RNA preservative. Filling can be done manually or by fluid handling robot. A sample container can be slipped over the reagent container and the seal can be engaged with the orifice of the reagent container so as to seal the reagent inside the compartment of the reagent container.

Sample containers and reagent containers can be made of any appropriate materials, including polymers, such as polypropylene and polyethylene.

### Examples

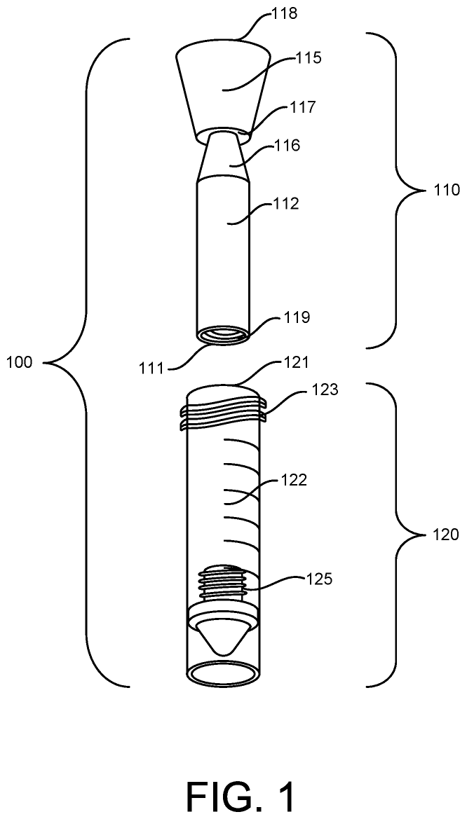

Referring to FIG. 1, sample container 100 includes first container 110 and second container 120. First container 110 includes an orifice 111 communicating with a first internal space 112 and comprising threads 119 that mate with seal 125; and a sleeve 115 attached to first container 110 at a tapered end 116 opposite first orifice 111. Sleeve 115 includes openings 117 (e.g., a drain) that allow a liquid deposited in a top opening 118 of the sleeve (e.g., mouth) to pass through. Sleeve 115 can be funnel-shaped to direct the flow of liquid deposited in it. Second container 120 includes an orifice 121 communicating with an internal space 122. Inside space 122 is disposed a seal 125. Seal 125 is configured to removably seal orifice 111 when engaged with orifice 111. First container 110 is configured to fit within second container 120. Container 120 also includes threads 123 to fit a screw cap.

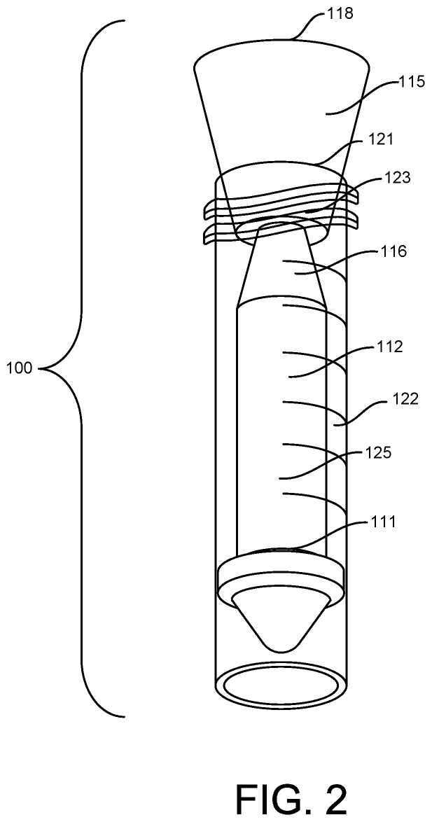

Referring to FIG. 2, the pieces are configured such that, when assembled, orifice 111 mates with and is removably sealed by seal 125, and such that the sleeve 115 fits within second orifice 121 such that fluid deposited into a mouth of sleeve 115 collects into internal space 122. In assembled form, internal space 112 contains a reagent, typically an aqueous solution, that is contained in the space by the seal. Furthermore, first container 110 and second container 120 have dimensions such that when they are mated, there is space remaining in internal space 121 to accept liquid deposited into mouth 118 of sleeve 115.

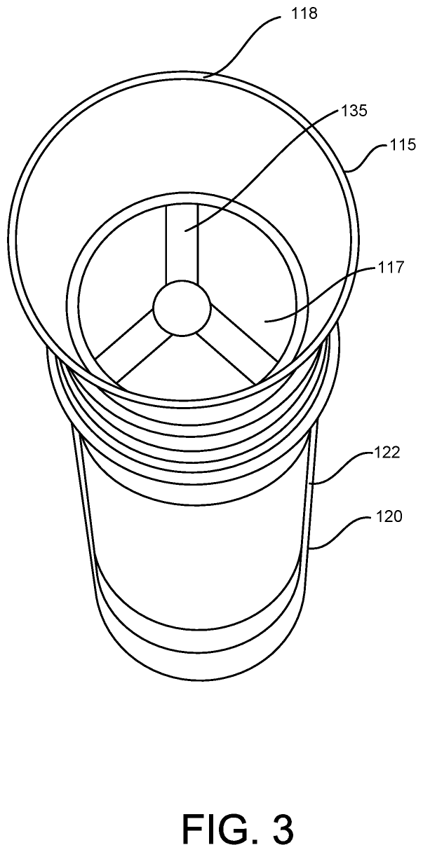

Referring to FIG. 3, sleeve 115 can be funnel or cone shaped. It comprises an opening 118 into which a sample can be introduced, and apertures or holes or passages 117 through which a sample introduced at opening 118 can pass into internal space 122 of second container 120.

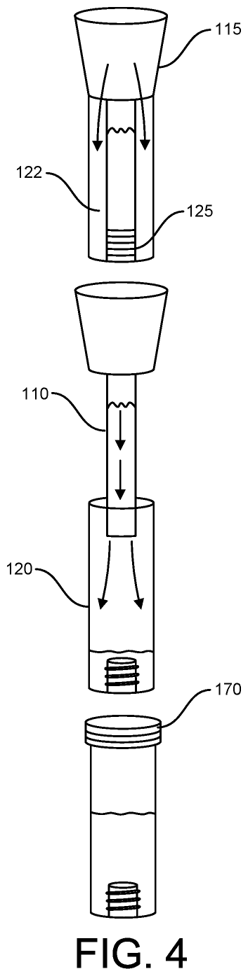

Referring to FIG. 4, in use, a fluid, such as saliva, is deposited into the mouth of sleeve 115 (1). Typically, fluid will flow, by force of gravity, around the outside of the shaft of container 110 into internal space 122. Then, orifice 111 of first container 110 is disengaged from seal 125 (2). This can be done, for example, by unscrewing the pieces, if they are sealed by a screw seal, or by twisting, if they are sealed by, for example, a plug forming a friction fit. Unsealing first container 110 releases aqueous solution 150 from the compartment of first container 110 into internal space 122 of second container 120. Second container 120 is then capped by cap 170 (3). Liquids insider second container 120 can then be mixed, for example, by shaking.

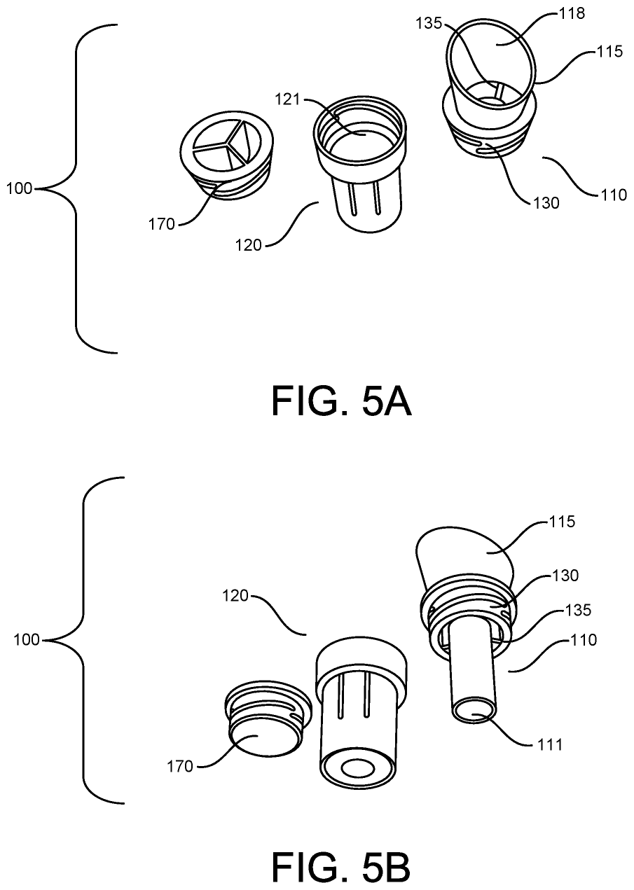

Referring to FIGS. 5A and 5B, in another embodiment, container 100, comprises a first container 110 and a second container 120. Opening 111 of container 110 mates with and sealed by a plug inside second container 120. The seal can be undone by twisting or to pulling first container 110 from second container 120. First container 110 also includes threads 130 that allow it to screw into orifice 121 of second container 120, which also has threads that mate with the threads on first container 110. Sleeve 115 is attached to container through ribs 135, which allows liquid deposited into orifice 118 of sleeve 115 to drip between the ribs 135 and into second container 120. After deposit, first container 110 can be unscrewed from second container 120, opening the plug and allowing reagent stored in the internal compartment of first container 110 to mix with the deposited liquid. Cap 170 is configured to screw into threads of opening 121 of container 120 after first container 110 is removed, thereby sealing second container 120 for transport.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A collection device comprising a first container positioned inside a second container, wherein the first container is releasably sealed by the second container, and wherein the first container comprises a sleeve that fits into an opening in the second container and that is configured to transmit fluid received into the sleeve into the second container, and wherein first container comprises a reagent, e.g., a liquid.

2. The collection device of claim 1, wherein, when an orifice in the first container is oriented toward gravity, unsealing the first container releases the reagent into the second container.

3. The collection device of claim 1, wherein the first container and second container are tube-shaped.

4. The collection device of claim 3, wherein the first tube has a tapered end.

5. The collection device of claim 4, wherein the sleeve is attached to the tapered end.

6. The collection device of claim 3, wherein the first container has an external diameter less than an internal diameter of the second container.

7. The collection device of claim 6, wherein the first container has an external diameter of about 10 mm to 20 mm and the second container has an internal diameter of about 15 mm to 30 mm.

8. The collection device of claim 1, wherein the sleeve is configured as a funnel.

9. The collection device of claim 1, wherein the second container comprises threads configured to screw into a top of the first container.

10. The collection device of claim 1, wherein the second container comprises a screw protruding from a bottom the second container, wherein the screw comprises threads configured to screw into internal threads in a top of the first container.

11. The collection device of claim 1, wherein the second container comprises a stopper configured to stop an orifice in the first container.

12. The collection device of claim 1, wherein the first container has a volume of about 2 ml to about 20 ml and the second container has a volume of about 7 ml to about 45 ml.

13. The collection device of claim 1, wherein the sleeve is attached to the first container through ribs, wherein spaces between ribs allow sample deposited into the sleeve to pass into the second container.

14. The collection device of claim 1, wherein the second container comprises an open end comprising internal or external threads for sealing with a screw cap.

15. The collection device of claim 1, wherein the first container and/or the second container comprise a plastic.

16. The collection device of claim 15, wherein the plastic comprises polypropylene or polyethylene.

17. The collection device of claim 1, wherein the reagent comprises an RNA preservative.

18. The collection device of claim 17, wherein the RNA preservative is selected from one or more of formalin, sulfate (e.g., ammonium sulfate), isothiocyanate (e.g., guanidinium isothiocyanate) and urea.

19. A kit comprising:
(a) a first container comprising:
(i) a first orifice communicating with a first internal space; and
(ii) a sleeve attached to the first container at an end opposite the first orifice, and wherein a sample deposited in the sleeve can pass through the sleeve; and
(b) a second container comprising:
(i) a second orifice communicating with a second internal space; and
(ii) a seal disposed within the second internal space, wherein the seal is configured to removably seal the first orifice when engaged therewith.

20. A collection device comprising:
(a) a first container comprising a first orifice communicating with a first internal space; and a sleeve configured to transmit a sample; and
(b) a second container comprising a second orifice communicating with a second internal space, and a seal disposed within the second internal space; wherein
the first container has dimensions to both fit into the second orifice and be sealed by the seal, and to position the sleeve to transmit fluid received into the sleeve into the second container.

21. The container of claim 20, wherein the sleeve is configured as a funnel having a wider end and a narrower end, wherein the narrower end is positioned proximal to the second orifice and the wider end is positioned distal to the orifice.

22. A kit comprising:
(a) a first container comprising:
(i) a first container comprising a first orifice communicating with a first internal space; and
(ii) a sleeve attached to the first container at an end opposite the first orifice, and wherein a sample deposited in the sleeve can pass through the sleeve; and
(b) a second container comprising:
(i) a second orifice communicating with a second internal space;
(ii) a seal disposed within the second internal space, wherein the seal is configured to removably seal the first orifice when engaged therewith; and
wherein the first container is configured to fit within the second container such that:
(1) the first orifice mates with and is removably sealed by the seal;
(2) the sleeve fits within the second orifice; and
(3) fitting the first container into the second container leaves space in the second internal space to hold sample deposited into the sleeve.

23. The kit of claim 22, further comprising a second cap configured to close the second orifice.

24. The kit of claim 22, wherein the seal engages the first container within the second internal space.

25. The kit of claim 22, wherein the seal does not engage the first container outside of the first internal space.

26. The kit of claim 22, wherein the seal is positioned distal from the second orifice.

27. A method comprising:
a) providing a collection device of claim 1 or 19;
b) depositing a sample into the second container through the sleeve; and
c) unsealing the first container from the second container, thereby contacting the reagent with the sample.

28. The method of claim 27, wherein the sample comprises saliva.

29. The method of claim 28, wherein depositing comprises spitting.

30. The method of claim 27, further comprising, after unsealing, sealing the second container.

31. The method of claim 30, wherein sealing the second container comprises attaching a screw cap to a top of the second container.

32. The method of claim 30, comprising, after sealing the second container, mixing the reagent and the sample in the second container.

33. The method of claim 30, comprising, after sealing the second container, depositing the second container in a mailing container and transmitting the mailing container via common carrier.

34. A method comprising:
(a) depositing a reagent through an orifice into an internal space of a first container; and
(b) sealing the orifice with a seal formed in an internal space of a second container;
wherein the second container has a greater volume than the first container.

35. The method of claim 34, wherein the reagent comprises a liquid, e.g., an aqueous liquid.

36. The method of claim 34, wherein the reagent comprises an RNA preservative.

37. The method of claim 34, wherein the first container has a volume between about 2 ml to about 20 ml.

38. The method of claim 34, wherein the second container has a volume between about 7 ml to about 45 ml.

39. The method of claim 34, wherein the seal comprises a screw, and sealing comprises screwing the second container into the orifice of the first container.

40. The method of claim 34, wherein the seal comprises a stopper, and sealing comprises inserting the stopper into the orifice of the first container.

41. The method of claim 34, wherein the first container and the second container comprise cylindrical portions, and sealing comprises sliding the cylindrical portion of the second contain over the cylindrical portion of the first container.

42. The method of claim 34, wherein the first container further comprises a sleeve disposed behind the orifice on the first container, wherein the sleeve has openings at both ends and, wherein sealing further comprises inserting a proximal end of the sleeve into an orifice of the second container, such that an opening in a distal end of the sleeve communicates through an opening in the proximal end of the sleeve with an internal space of the second container.

43. The method of claim 42, wherein the sleeve has a funnel shape with a narrower end positioned proximal to the first container and the wider end positioned distal to the first container.

44. The method of claim 34, wherein the seal engages the first container within the internal space of the first container.

45. The method of claim 34, wherein the seal does not engage the first container outside of the internal space of the first container.

46. The method of claim 34, wherein the seal is positioned distal from an opening to the internal space of the second container.

47. A collection device comprising:
(a) a first container comprising:
(i) at a first end, an opening to a cavity, wherein the cavity contains a liquid; and
(ii) at a second end, a funnel-shaped sleeve, wherein the sleeve has a wider mouth and a narrower mouth, and is attached to the first container by one or a plurality of ribs; and
(b) a second container comprising:
(i) an opening to a cavity, wherein the cavity comprises a cap; wherein:
(I) the first container is positioned within the cavity of the second container;
(II) the cap releasably closes the opening of the first container; and
(III) the narrower mouth of the sleeve is positioned within the opening of the second container and the wider mouth of the sleeve is positioned outside of the opening of the second container.
